(19) United States
(12) Patent Application Publication
(10) Pub. No.: US 2009/0131711 A1
(43) Pub. Date: May 21, 2009

(54) SINGLE-STAGE ESTERIFICATION OF OILS AND FATS

(75) Inventor: Udaya Wanasundara, Saskatoon (CA)

Correspondence Address: FASKEN MARTINEAU DUMOULIN, LLP, 2900 - 550 Burrard Street, VANCOUVER, BC, V6C 0A3, CA

(73) Assignee: POS PILOT PLANT CORP., Saskatoon (CA)

(21) Appl. No.: 11/997,263

(22) PCT Filed: Jul. 27, 2006
(86) PCT No.: PCT/CA06/01238

§ 371 Date: Dec. 3, 2008

Related U.S. Application Data
(60) Provisional application No. 60/703,447, filed on Jul. 29, 2005.

(51) Int. Cl. C07C 67/48

(52) U.S. Cl. 560/248

(57) ABSTRACT

A process for producing alkyl esters useful as bio-fuels and/or lubricants. An alkyl ester product produced by the process. The process comprises a single-stage reaction for esterifying a de-watered glyceride-containing feedstock with an anhydrous short chain alcohol in the presence of a basic esterification catalyst to produce a reaction product comprising alkyl esters and a reaction by-product comprising glycerol-containing substances and the catalyst-containing alcohol. The single-stage esterification reaction is conducted within a temperature and negative-pressure controllable vessel. The alkyl ester product is separated from the re-action by-product and may be further de-watered and/or purified. The catalyst-containing alcohol may be separated from the reaction by-product, de-watered and reused. The glycerol-containing substances may be separated from the reaction by-product and further purified. Useful glyceride-containing feedstocks include those prepared from plant or animal or fish materials, particularly those produced from seeds of mustard, canola, soybean, corn, cotton, flax and palm.

### TECHNICAL FIELD

This invention relates to the esterification of oils and fats to form products useful, for example, as fuels or fuel additives or lubricants. More particularly, the invention relates to such processes that can be used for the production of so-called biodiesel fuels as well as other products.

### BACKGROUND ART

Diesel fuels derived from refining crude oil and other forms of petroleum raw feed stocks are relatively inexpensive to produce and therefore are widely used to power internal combustion engines for numerous light- and heavy-duty transportation and stationary industrial applications. However, combustion of such diesel fuels typically produces significant amounts of gaseous pollutants in the attendant exhaust gases including nitric oxide, nitrogen dioxide, sulphur dioxide, carbon monoxide, and hydrocarbons. Furthermore, temperatures achieved in most diesel-fired engines are not sufficiently high enough to provide complete combustion of diesel fuels thereby generating significant amounts of particulate emissions in the form of black soot or smoke from the engine exhaust stacks. Consequently, numerous regulatory agencies have established and promulgated long-term escalating emission standards for reducing gaseous and particulate matter in diesel engine exhaust gases.

Various strategies have been implemented to achieve current and pending regulatory standards for emissions from diesel engines, including: (a) modification of engine designs to provide more efficient and complete fuel combustion, (b) extending and modifying refining processes to provide more highly-purified and cleaner burning petroleum-derived diesel fuels, (c) development of chemically synthesized additives for mixing into refined diesel fuels to improve their combustion and emissions properties, and (d) assessment of alternative raw feed stocks such as animal and plant-derived fats and oils for the production of fuels commonly called “biofuels” or “biodiesel”, that have similar thermal combustion and power-generating properties to petroleum-based diesel fuels.

There is considerable interest in the use of plant-derived oils and animal fats as feedstocks for production of biodiesel fuels for use in diesel engines because these alternative fuels are significantly cleaner burning, i.e., their exhaust gases contain significantly reduced levels of gaseous and particulate emissions, compared to petroleum-based diesel fuels. Biodiesel fuels are typically produced by processes which involve esterification of triglycerides and free fatty acids present in the feedstocks with short-chain alcohols to form alkyl esters of the fatty acids as the primary products as well as commercially useful by-products such as glycerol. The esterification reaction is generally described as:

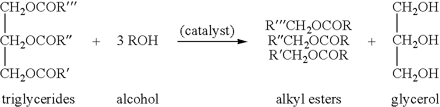

There are three basic processes known for conversion of oil and fat feedstocks into biodiesel fuels:
1. base-catalyzed esterification of the feedstocks,
2. direct acid-catalyzed esterification of the feedstocks, and
3. conversion of triglyceride compounds in the feedstocks to fatty acids followed by esterification of the fatty acids.

It is known that hydrogenated and unhydrogenated animal fats, rendered fats, vegetable oils, fish oils, spent restaurant grease, and waste industrial frying oils are suitable feedstocks for production of biodiesel fuels by such esterification processes. The simplest and therefore most common process is the base-catalyzed reaction because it does not require exotic catalysts such as those disclosed in U.S. Pat. Nos. 5,508,457, 5,525,126 and 6,878,837, or system configurations such as those disclosed in U.S. Pat. Nos. 6,174,501, 6,187,939 and 6,887,283, and can be performed with relatively lower temperatures, pressures and reaction/synthesis times. However, in order to produce a sufficiently purified biodiesel fuel or fuel additive suitable for use with current diesel engine designs, Dmytryshyn et. al.(2004, Bioresource Technology 92: 55-64) teach that the base-catalyzed process must be conducted in two stages as exemplified in FIG. 1 of the accompanying drawings wherein the first stage is the reaction of the feedstock with a short-chain alcohol containing a basic esterification catalyst (e.g., an alkoxide or hydroxide of potassium or sodium) to produce a mixture of crude alkyl esters, mono-, di-, and triglycerides followed by separation of the crude alkyl esters from the triglycerides. The second stage is the further purification of the crude alkyl esters with additional volume(s) of short-chain alcohol containing additional basic esterification catalyst, after which the purified alkyl esters are washed to remove the alcohol, then dewatered and filtered. The final alkyl ester products are relatively pure, i.e., more than 95%, but typically provide 65% or less conversion efficiencies from the source materials present in the feedstocks due to losses of product during purification at completion of stage 1 and stage 2. Therefore, even though the base-catalyzed processes are more economic at present than the alternative methods for producing biodiesel fuels, the need for extensive purification and dewatering steps adds to the complexity, time requirements and costs of the processes relative to petroleum-derived diesel fuels.

### DISCLOSURE OF THE INVENTION

The exemplary embodiments of the present invention, at least in preferred forms, are directed to processes for producing alkyl esters useful as biodiesel fuels and lubricants from a feedstock containing glyceride-containing substances, said alkyl esters produced in a single-stage alkylation reaction of the glycerides in said feedstock with an alcohol containing therein a reaction catalyst.

According to a preferred embodiment of the present invention, there is provided a process for producing alkyl esters from a feedstock containing glyceride-containing substances in a single-stage esterification reaction wherein the first step is dewatering of the feedstock after which, the dewatered feedstock is combined with an alcohol (preferably anhydrous or dewatered from a previous stage of the process) containing therein a reaction catalyst for esterification of the glycerides thereby producing alkyl esters in a single-stage alkylation reaction. The single-stage alkylation reaction provides a reaction product comprising alkyl esters suitable for use as biodiesel fuels and lubricants, and a reaction by-product comprising reaction catalyst-containing alcohol, glycerols and glycerol-containing substances. The reaction product is preferably de-watered and purified after separation from the reaction mixture.

According to one aspect, the glyceride-containing feedstock is an oil-based feedstock prepared from plant materials. In a preferred form, the plant-based oil-containing feedstock is a material prepared from the seeds of a plant selected from the group comprising brassicas, legumes, maize, cotton, flax and palms. Non-limiting examples of useful plant seeds include mustard, canola, soybean, corn, cotton, flax and oil-seed palms.

According to another aspect, the glyceride-containing feedstock is an oil-based feedstock prepared from animal materials. In a preferred form, the animal-based oil-containing feedstock is a material prepared from rendered animal fats.

According to yet another aspect, the glyceride-containing feedstock is an oil-based feedstock selected from the group comprising waste restaurant frying oils, waste vehicular and/or locomotive and/or marine engine oils, and waste industrial lubricants and oils, said waste oils and lubricants characterized by a low free fatty acid content.

According to another preferred embodiment of the present invention, there is provided a process comprising a single-stage esterification reaction for producing an alkyl ester reaction product from a glyceride-containing feedstock. The single stage reaction comprises contacting a dewatered glyceride-containing feedstock with an alcohol containing therein a suitable reaction catalyst. The reaction is preferably conducted in a pressure-resistant vessel wherein temperature and negative pressure are controllably maintainable for a period of time sufficient to convert said glycerides into an alkyl ester reaction product and a reaction by-product mixture comprising glycerols and/or glycerol-containing substances and said alcohol, after which, the reaction product is separated from the reaction by-product.

According to one aspect, the alcohol is preferably a short-chain alcohol. It is preferred that the alcohol is selected from the group comprising methanol, ethanol, propanol and butanol.

According to another aspect, the reaction temperature is preferably selected from the range of 50° C. to 100° C. It is preferable that the negative pressure is selected from the range of 99 mmHg(A) to 760 mmHg(A).

According to yet another aspect, the alky ester reaction product separated from the reaction by-product, is de-watered. The de-watered alky ester reaction product is purified by contacting the reaction product with an adsorbent material. In a preferred form, the adsorbent material is selected from the group comprising silicas and clays. It is preferable that the adsorbent material is a silica.

According to a further aspect, the catalyst-containing alcohol is separated from the reaction by-product mixture. It is preferred that the separated catalyst-containing alcohol is de-watered and recycled for use in another single-stage esterification reaction.

According to a yet further aspect, the glycerols and glycerol-containing substances comprising the reaction by-product mixture are separated from said mixture. Said separated glycerols and glycerol-containing substances may optionally be further separated and purified.

According to yet another preferred embodiment, there is provided an alkyl ester product produced from a glyceride-containing feedstock by a single-step esterification reaction of the present invention. The glyceride-containing feedstock is preferably an oil-based feedstock prepared from materials selected from the group comprising plant materials, animal materials and fish materials. In a preferred form, the plant materials are selected from the group comprising brassica seeds, legumes seeds, maize seeds, mallow seeds, flax seeds and palm nuts.

According to a further preferred embodiment, there is provided a glycerol-containing product produced from a glyceride-containing feedstock by a single-step esterification reaction of the present invention.

Other exemplary embodiments provide a process for producing alkyl esters in a single-stage esterification reaction of a dewatered feedstock containing therein glyceride-containing substances, thereby providing a reaction product comprising alkyl esters and a reaction by-product comprising glycerol-containing substances.

### BRIEF DESCRIPTION OF THE DRAWINGS

The present invention will be described in conjunction with reference to the following drawing, in which:

FIG. 1 is a schematic flow diagram illustrating a prior art process; and

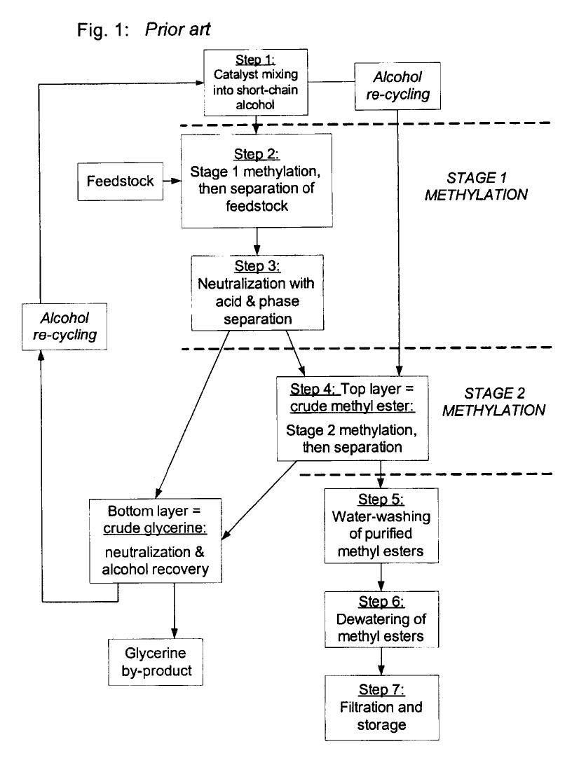

FIG. 2 is a schematic flow diagram illustrating an embodiment of the present invention.

### BEST MODES FOR CARRYING OUT THE INVENTION

The present invention provides a process for producing alkyl esters from a feedstock containing oils or fats, in a single-stage esterification reaction performed under controlled temperature and pressure conditions that are significantly lower than those disclosed and taught in the prior art. The first step in the process of the present invention is to dewater the feedstock by raising the temperature of the feedstock to within the range of 50° C. to 100° C. after which, the heated feedstock is sprayed into a vessel whereto a negative pressure in the range of 99 mmHg(A) to 760 mmHg(A) has been applied and wherein the temperature of the heated feedstock is maintained while the feedstock is agitated to maintain a turbulent flow until the feedstock is substantially free of water, i.e., it contains less than 1% water (v/v). Alternatively, the feedstock may be added into a temperature- and pressure-controllable vessel, wherein it is agitated to maintain a turbulent flow under a negative pressure in the range of 99 mmHg(A) to 760 mmHg(A) while its temperature is raised to within the range of 50° C. to 100° C. and then maintained in a turbulent flow at said temperature until the feedstock is substantially free of water. The dewatered feedstock is cooled prior to mixing with a short-chain alcohol containing a basic esterification catalyst thereby initiating an exothermic reaction whereby the triglycerides present in the feedstock are alkylated in a single stage to produce alkyl esters suitable for use as biodiesel fuels and lubricants. It is preferable that the single-stage esterification reaction is conducted within temperature- and pressure-controllable vessels provided with mixing equipment capable of producing and maintaining a turbulent flow so that the temperature and agitation of the feedstock can be precisely controlled during the esterification reaction. Temperatures may be precisely controlled during the exothermic reaction by providing said vessels with external water jackets or alternatively, with cooling coils within the vessels. There are also provided methods for purifying the alkyl esters produced by the process of the present invention for single-stage esterification of a dewatered feedstock. The process of the present invention additionally produces as reaction by-products such as crude glycerol and soaps which if so desired, may be further purified for other types of use.

The process of the present invention is suitable for use with any feedstock containing plant-derived oils such as oils obtained for example from crushing seeds from mustard, canola, soybean, corn, cotton, flax, or palm and other plants producing seeds with high oil-content. Alternatively, the process of the present invention is also suitable for use with feedstocks containing fish oils or animal fats including rendered fats or tallow. Furthermore, the process of the present invention can also be used with feedstocks comprising waste restaurant or industrial grease and/or frying oils containing very low levels of free fatty acids.

The alcohol used in the process of the present invention is preferably a dewatered short chain alcohol within the range of C₁₋₄ containing less than 1% water (v/v). It is preferable to use absolute methanol; however, absolute ethanol, absolute propanol and absolute butanol can be used satisfactorily in the process of the present invention. It is important to thoroughly mix a basic esterification catalyst into the alcohol prior to adding the alcohol-basic catalyst mixture into a dewatered feedstock. While the process of the present invention can be employed with any basic esterification catalyst mixed into the alcohol, it is preferred to use potassium hydroxide or alternatively, sodium hydroxide.

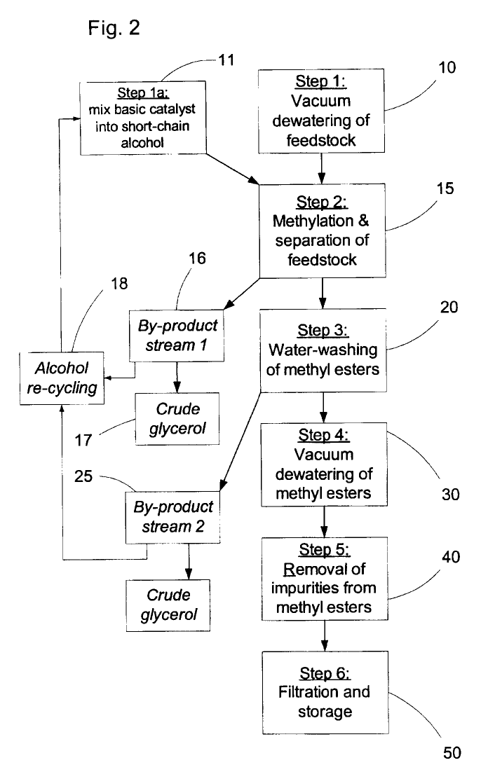

In one preferred embodiment of the present invention, the process illustrated in FIG. 2 comprises 6 steps wherein the step 1 as indicated by reference number 10, a volume of feedstock is dewatered by first heating it to a temperature of about 100° C., then spraying the heated feedstock into an water jacketed vessel wherein the inner temperature is maintained at 95° C.±5° C. under a vacuum-applied negative pressure of 110 mmHg(A). The feedstock is maintained in a constant turbulent flow by agitation at that temperature for at least 1 hr after which, the feedstock is cooled to approximately 40° C. while maintaining the turbulent flow and negative pressure within the insulated vessel. While the feedstock is being dewatered, a basic esterification catalyst, e.g., potassium hydroxide (KOH), is mixed into a dewatered short-chain alcohol e.g., absolute methanol as indicated by reference number 11 which is then added under negative pressure to the dewatered feedstock in the insulated vessel while the feedstock is controllably agitated thereby causing the esterification reaction as indicated by reference number 15 to proceed. The negative pressure in the insulated vessel should be released after the addition of the alcohol-basic esterification catalyst mixture. The esterification reaction is exothermic and therefore, it is important to maintain the temperature within the insulated vessel at a constant setting less than 100° C. and preferably within the range of 40° C. to 60° C. while the feedstock is maintained by agitation in a turbulent flow for at least 2 hours at atmospheric pressure after which, agitation is ceased to allow the methyl esters to separate into a top layer above a bottom layer containing the reaction by-products and alcohol-basic esterification catalyst mixture. After separation has been completed, the bottom layer as indicated by reference number 16 containing the reaction by-products is removed from the insulated vessel after which the alcohol may be recovered from the reaction by-products by distillation, and recycled as indicated by reference number 18, leaving a crude glycerol by-product as indicated by reference number 17 which may be further purified if so desired. The top layer containing the methyl ester reaction product is maintained at a constant setting of less than 75° C. preferably within the range of 40° C. to 50° C., while wash water is heated to temperature of about 95° C. The heated wash water is added to the methyl ester reaction product under negative pressure applied by vacuum to the insulated vessel after which, the negative pressure is released and then the water-methyl ester mixture as indicated by reference number 20 is agitated for at least 30 min at atmospheric pressure after which agitation is ceased. Separation is allowed to proceed for 4-6 hrs during which time the washed methyl esters form a top layer over a bottom layer of wash water thereby removing impurities from the methyl ester reaction product into the wash layer. The bottom wash layer mixture as indicated by reference number 25 is removed from the insulated vessel. Alternatively, the methyl ester and water phases can be separated by disc stack centrifugation (i.e., de-slugging) to reduce the time required for separation of the phases to approximately 1 hour. The purified methyl ester product is then dewatered mixture as indicated by reference number 30 by placing the insulated vessel under negative pressure of 110 mmHg(A) and controllably raising the temperature within the vessel to a constant temperature of about 95° C. for at least 30 min. At this step in the process of the present invention, the dewatered methyl ester product may be further purified by the addition of a silica-based or clay-based adsorbent as indicated by reference number 40 and then agitating the mixture to bind any trace impurities such as soaps, remaining mono- or di-glycerides, or trace metals to the adsorbent thereby further purifying the methyl esters. The adsorbent is removed from the methyl ester product by filtration after which, the methyl esters may be used as biodiesel fuels, additives or lubricants or alternatively, packaged and stored.

The examples presented below are included as embodiments of the present invention, but are not intended to limit the scope of the present invention.

### EXAMPLE 1

A 1,700 kg quantity of mustard oil was sprayed with a nozzle pressure of 10.0±5.0 psi into a temperature- and pressure-controlled water-jacketed reactor. After the mustard oil was added to the reactor, a negative pressure of 110 mmHg(A) was applied by vacuum, and the oil was dewatered by heating to and maintenance at 100° C.±5° C. under negative pressure while being maintained in a turbulent flow by agitation until there was no trace of bubbling at the oil surface after which, the oil was cooled to 50° C.±2° C.

While the oil was being dewatered, 340.0 kg of absolute methanol (20% w/w of the oil) was added to a stirred vessel and then 17.0 kg of KOH (1% w/w of the oil) was added while the methanol was vigorously agitated until the KOH was completely dissolved. The KOH-methanol mixture was added under vacuum to the 1,700 kg of dewatered oil maintained in a turbulent flow by agitation after which, the negative pressure within the reactor was broken with nitrogen gas which was used to flush and then maintain the headspace within the reactor. Addition of the KOH-methanol mixture to the dewatered oil initiated an exothermic esterification reaction and therefore, the temperature within the water-jacketed reactor was carefully maintained at 50° C.±2° C. during the reaction period. The esterification reaction proceeded for 4.5 hrs while the oil was maintained in a turbulent flow by agitation. At the end of this time period, agitation was ceased and the methylated mustard oil was maintained in the reactor for 2 hrs to allow the reaction mixture to separate into two phases comprising a top layer containing the methyl ester reaction product above a bottom layer containing the reaction by products, i.e., spent methanol and crude glycerol.

After the phase separation was complete, the bottom layer containing the reaction by-products was removed from the reaction vessel after which the temperature of the retained methyl ester product was adjusted to 50° C. Then, 500 L of water heated to 95° C. was added to the methyl ester phase under vacuum after which, the negative pressure was released and the mixture was agitated for 30 min at atmospheric pressure to wash water-soluble impurities out of the methyl ester reaction product after which, agitation was stopped. The mixture was allowed to separate over an 8-hr period into 2 phases comprising a top layer containing washed methyl ester reaction product above a bottom layer containing water-soluble by-products removed from the methyl ester reaction product.

After the second phase separation was complete, the bottom layer was removed. A negative pressure of 110 mmHg(A) was then applied by vacuum to the washed methyl ester reaction product remaining in the reactor while the temperature of the reaction product was raised to 95° C.±5° C. under agitation to dewater the washed methyl ester water product. After dewatering was completed, the temperature of the methyl ester reaction product was adjusted to 60° C.±3° C. after which, 2% (w/w) TriSyl® 615 adsorbent (TriSyl is a registered trademark of W.R. Grace & Co.) was added to the reaction product and mixed for 30 min to remove any remaining impurities. The adsorbent was removed from the methyl ester reaction product by recycling the methyl esters through a pressure filter apparatus until clarity was achieved. The methyl ester reaction product was then packaged and analyzed.

### Results:

The data in Table 1 show that the process of the present invention provided a 99% conversion of the triglyceride compounds present in mustard oil into methyl esters, the purity of the final methyl ester product was 98.95%, and the recovery was 91.8%, i.e., 1,700 kg of mustard oil yielded 1,560 kg of methyl ester product containing 1,531 kg methyl esters. The data in Table 2 show that the 91.8% of the starting raw material (i.e., crude mustard oil) was recovered and purified methyl ester reaction product.

TABLE 1
Analysis of mustard methyl ester reaction product.

| Component | Concentration | |
|---|---|---|
| Soap | 0 | ppm |
| Acid value | 0.12 | mg KOH/g |
| Karl Fisher moisture value | 666 | ppm |
| Free glycerol | <0.001% | |
| Total glycerol | <0.01% | |
| Total methylated fatty acid content | 989.5 | mg/g product |

Individual methylated fatty acids

| | | |
|---|---|---|
| C14 - Myristic acid | 0.5 | mg/g product |
| C16 - Palmitic acid | 29.0 | mg/g product |
| C16:1n7 - Palmitoleic acid | 1.5 | mg/g product |
| C17:0 Margaric acid | — | mg/g product |
| C18 - Steric acid | 14.4 | mg/g product |
| C18:1n9 - Oleic acid | 216.2 | mg/g product |
| C18:1 - Octadecenoic acid | 11.1 | mg/g product |
| C18:2n6 - Linoleic acid | 202.7 | mg/g product |
| C18:3n3 - alpha-linoleic acid | 110.6 | mg/g product |
| C20 - Arachidic acid | 8.3 | mg/g product |
| C20:1n9 - Eicosenoic acid | 118.3 | mg/g product |
| C20:2n6 - Eicosadienoic acid | 9.7 | mg/g product |
| C20:3n3 - Mead's acid | 1.7 | mg/g product |
| C22 - Behenic acid | 4.6 | mg/g product |
| C22:1n9 - Erucic acid | 231.6 | mg/g product |
| C22:2n6 - Docosadienoic acid | 1.7 | mg/g product |
| C21:5n3 - Heneicosapentaenoic acid | 4.2 | mg/g product |
| C22:4n6 - Docosatetraenoic acid | 1.1 | mg/g product |
| C22:5n6 - Docosapentaenoic acid | 0.6 | mg/g product |
| C24 - Lignoceric acid | 2.8 | mg/g product |
| C24:1n9 - Nervonic acid | 16.5 | mg/g product |
| Other fatty acids | 2.4 | mg/g product |

TABLE 2
Mass Balance calculation.

| Input: | 1,700 kg mustard oil |
|---|---|
| Output: | 1,560 kg washed and purified mustard methyl |
| | ester reaction product |
| Percent recovery: | 91.8% |

### EXAMPLE 2

A 500 g quantity of canola oil was added into temperature- and pressure-controlled water-jacketed reactor 2 L all Stainless Steel Pressure Reactor (Parr Instrument Company, Moline, Ill., USA). The canola oil was dried by a negative pressure of 110 mmHg(A) applied by vacuum, and the oil was dewatered by heating to and maintenance at 100° C.±5° C. under negative pressure while being maintained in a turbulent flow by agitation until there was no trace of bubbling at the oil surface after which, the oil was cooled to 50° C.±2° C.

While the oil was being dewatered, 100 g of absolute methanol (20% w/w of the oil) was added to a stirred vessel and then 5 g of KOH (1% w/w of the oil) was added while the methanol was vigorously agitated until the KOH was completely dissolved. The KOH-methanol mixture was added under vacuum to the 500 g dewatered oil maintained in a turbulent flow by agitation after which, the negative pressure within the reactor was broken with nitrogen gas which was used to flush and then maintain the headspace within the reactor. Addition of the KOH-methanol mixture to the dewatered oil initiated an exothermic esterification reaction and therefore, the temperature within the water-jacketed reactor was carefully maintained at 50° C.±2° C. during the reaction period. The esterification reaction proceeded for 4 hrs while the oil was maintained in a turbulent flow by agitation. At the end of this time period, agitation was ceased and the methylated canola oil was transferred to a separation funnel where it was maintained for 18 hrs to allow the reaction mixture to separate into two phases comprising a top layer containing the methyl ester reaction product above a bottom layer containing the reaction by products, i.e., spent methanol and crude glycerol.

After the phase separation was complete, the bottom layer containing the reaction by-products was removed from the separation funnel after which, the top methyl ester layer was removed into a separate container. The temperature of the methyl ester product was adjusted to about 75 after which water heated to 95° C. was added to the methyl ester phase until a ratio of 85:10 methyl ester:water was reached. The mixture was then vigorously agitated for 10 min and then centrifuged at 4,200 rpm for 10 min to separate the mixture into 2 phases comprising a top layer containing washed methyl ester reaction product above a bottom layer containing water-soluble by-products removed from the methyl ester reaction product. The top layer containing the washed methyl ester product was decanted and transferred to rotary evaporator flasks wherein any remaining water was removed.

After dewatering was completed, the temperature of the methyl ester reaction product was adjusted to 60° C.±3° C. after which, 0.5 2% (w/w) TriSyl® 615 adsorbent was added to the reaction product and mixed for 15 min to remove any remaining impurities. The adsorbent was removed from the methyl ester reaction product by recycling the methyl esters through a pressure filter apparatus until clarity was achieved. The methyl ester reaction product was then packaged and analyzed.

### Results:

The data in Tables 3 and 4 show that the process of the present invention provided a 99% conversion of the triglyceride compounds present in canola oil into methyl esters, the purity of the final methyl ester product was 100%, and the recovery was 85% i.e., 500 g of canola oil yielded 425 g of methyl ester product containing 421 g methyl esters.

TABLE 3
Analysis of canola methyl ester reaction product.

| Component | Concentration | |
|---|---|---|
| Soap | 0 | ppm |
| Acid value | 0.03 | mg KOH/g |
| Karl Fisher moisture value | 65 | ppm |
| Free glycerol | <0.01% | |
| Total glycerol | <0.10% | |
| Total methylated fatty acid content | 993.5 | mg/g product |

Individual methylated fatty acids

| | | |
|---|---|---|
| C14 - Myristic acid | 0.3 | mg/g product |
| C16 - Palmitic acid | 41.5 | mg/g product |
| C16:1n7 - Palmitoleic acid | 2.6 | mg/g product |
| C17:0 Margaric acid | 1.6 | mg/g product |
| C18 - Steric acid | 17.0 | mg/g product |
| C18:1n9 - Oleic acid | 558.7 | mg/g product |
| C18:1 - Octadecenoic acid | 30.1 | mg/g product |
| C18:2n6 - Linoleic acid | 119.6 | mg/g product |
| C18:3n3 - alpha-linoleic acid | 106.1 | mg/g product |
| C20 - Arachidic acid | 6.6 | mg/g product |
| C20:1n9 - Eicosenoic acid | 15.1 | mg/g product |
| C20:2n6 - Eicosadienoic acid | 0.6 | mg/g product |
| C20:3n3 - Mead's acid | — | mg/g product |
| C22 - Behenic acid | 3.6 | mg/g product |
| C22:1n9 - Erucic acid | 1.4 | mg/g product |
| C22:2n6 - Docosadienoic acid | — | mg/g product |
| C21:5n3 - Heneicosapentaenoic acid | — | mg/g product |
| C22:4n6 - Docosatetraenoic acid | — | mg/g product |
| C22:5n6 - Docosapentaenoic acid | — | mg/g product |
| C24 - Lignoceric acid | 1.2 | mg/g product |
| C24:1n9 - Nervonic acid | 2.0 | mg/g product |
| Other fatty acids | 5.4 | mg/g product |

TABLE 4
Mass Balance calculation.

| Input: | 500 g canola oil |
|---|---|
| Output: | 425 g washed and purified canola methyl |
| | ester reaction product |
| Percent recovery: | 85% |

### EXAMPLE 3

A 500 g quantity of soybean oil was added into temperature- and pressure-controlled water-jacketed reactor 2 L all stainless steel pressure Reactor (Parr Instrument Company, Moline, Ill., USA). The soybean oil was dried by a negative pressure of 110 mmHg(A) applied by vacuum, and then heating and maintaining the oil at 100° C.±5° C. under negative pressure while being maintained in a turbulent flow by agitation until there was no trace of bubbling at the oil surface after which, the oil was cooled to 50° C.±2° C.

While the soybean oil was being dewatered, 100 g of absolute methanol (20% w/w of the oil) was added to a stirred vessel and then 5 g of KOH (1% w/w of the oil) was added while the methanol was vigorously agitated until the KOH was completely dissolved. The KOH-methanol mixture was added under vacuum to the 500 g dewatered soybean oil maintained in a turbulent flow by agitation after which, the negative pressure within the reactor was broken with nitrogen gas which was used to flush and then maintain the headspace within the reactor. Addition of the KOH-methanol mixture to the dewatered oil initiated an exothermic esterification reaction and therefore, the temperature within the water-jacketed reactor was carefully maintained at 50° C.±2° C. during the reaction period. The esterification reaction proceeded for 4 hrs while the oil was maintained in a turbulent flow by agitation. At the end of this time period, agitation was ceased and the methylated soybean oil was transferred to a separation funnel where it was maintained for 18 hrs to allow the reaction mixture to separate into two phases comprising a top layer containing the methyl ester reaction product above a bottom layer containing the reaction by products, i.e., spent methanol and crude glycerol.

After the phase separation was complete, the bottom layer containing the reaction by-products was removed from the separation funnel after which, the top methyl ester layer was removed into a separate container. The temperature of the methyl ester product was adjusted to about 75 after which water heated to 95° C. was added to the methyl ester phase until a ratio of 85:10 methyl ester:water was reached. The mixture was then vigorously agitated for 10 min and then centrifuged at 4,200 rpm for 10 min to separate the mixture into 2 phases comprising a top layer containing washed methyl ester reaction product above a bottom layer containing water-soluble by-products removed from the methyl ester reaction product. The top layer containing the washed methyl ester product was decanted and transferred to rotary evaporator flasks wherein any remaining water was removed.

After dewatering was completed, the temperature of the methyl ester reaction product was adjusted to 60° C.±3° C. after which, 2% (w/w) TriSyl® 615 adsorbent was added to the reaction product and mixed for 15 min to remove any remaining impurities. The adsorbent was removed from the methyl ester reaction product by recycling the methyl esters through a pressure filter apparatus until clarity was achieved. The methyl ester reaction product was then packaged and analyzed.

### Results:

The data in Tables 5 and 6 show that the process of the present invention provided a 100% conversion of the triglyceride compounds present in soybean oil into methyl esters, the purity of the final methyl ester product was 100%, and the recovery was 88.6%, i.e., 500 g of soybean oil yielded 443 g of methyl ester product containing 443 g methyl esters.

TABLE 5
Analysis of soybean methyl ester reaction product.

| Component | Concentration | |
|---|---|---|
| Soap | 0 | ppm |
| Acid value | 0.04 | mg KOH/g |
| Karl Fisher moisture value | 74 | ppm |
| Free glycerol | <0.01% | |
| Total glycerol | <0.08% | |
| Total methylated fatty acid content | 1,000.0 | mg/g product |

Individual methylated fatty acids

| | | |
|---|---|---|
| C14 - Myristic acid | 0.7 | mg/g product |
| C16 - Palmitic acid | 103.5 | mg/g product |
| C16:1n7 - Palmitoleic acid | 1.0 | mg/g product |
| C17:0 Margaric acid | 1.0 | mg/g product |
| C18 - Steric acid | 45.0 | mg/g product |
| C18:1n9 - Oleic acid | 218.6 | mg/g product |
| C18:1 - Octadecenoic acid | 12.7 | mg/g product |
| C18:2n6 - Linoleic acid | 530.7 | mg/g product |
| C18:3n3 - alpha-linoleic acid | 75.1 | mg/g product |
| C20 - Arachidic acid | 3.7 | mg/g product |
| C20:1n9 - Eicosenoic acid | 2.8 | mg/g product |
| C20:2n6 - Eicosadienoic acid | — | mg/g product |
| C20:3n3 - Mead's acid | — | mg/g product |
| C22 - Behenic acid | 3.4 | mg/g product |
| C22:1n9 - Erucic acid | — | mg/g product |
| C22:2n6 - Docosadienoic acid | — | mg/g product |
| C21:5n3 - Heneicosapentaenoic acid | — | mg/g product |
| C22:4n6 - Docosatetraenoic acid | — | mg/g product |
| C22:5n6 - Docosapentaenoic acid | — | mg/g product |
| C24 - Lignoceric acid | 0.8 | mg/g product |
| C24:1n9 - Nervonic acid | — | mg/g product |
| Other fatty acids | 3.8 | mg/g product |

TABLE 6
Mass Balance calculation.

| Input: | 500 g soybean oil |
|---|---|
| Output: | 443 g washed and purified soybean methyl |
| | ester reaction product |
| Percent recovery: | 88.6% |

1. A process for producing alkyl esters useful as biofuels and lubricants from a glyceride-containing feedstock, said process comprising the steps of:
dewatering the feedstock by applying a negative pressure of about 110 mmHg(A) for about 60 minutes while maintaining the feedstock at a temperature within the range of about 90° C. to about 100° C., and then cooling the feedstock to about 40° C. while maintaining the negative pressure until the water content of the feedstock is less than 1% (w/w);
esterifying the dewatered feedstock with an anhydrous short chain alcohol selected from the group consisting of methanol, ethanol propanol and butanol in the presence of a basic esterification catalyst;
separating alkyl ester products and glycerol-containing by-products thereby formed;
and purifying said alkyl ester products, said esterification step carried out as a single-stage reaction.

2. A process according to claim 1 wherein said alkyl ester products are purified by contacting said alkyl ester products with an adsorbent material.

3. A process according to claim 2 wherein said adsorbent material is selected from the group comprising silicas and clays.

4. A process according to claim 1 wherein said glyceride-containing feedstock comprises oils extracted from materials selected from the group consisting of plants, animals and fish.

5. A process according to claim 4 wherein said plant materials are selected from the group consisting of brassicas, legumes, maize, mallows, flax and palms.

6. A process according to claim 5 wherein said plant materials are seeds selected from the group consisting of mustard seeds, canola seeds and rape seeds.

7. A process according to claim 5 wherein said plant materials are soybean seeds.

8. A process according to claim 5 wherein said plant materials are corn seeds.

9. A process according to claim 5 wherein said plant materials are cotton seeds.

10. A process according to claim 5 wherein said plant materials are flax seeds.

11. A process according to claim 5 wherein said plant materials are palm nuts.

12. A process according to claim 4 wherein said glyceride-containing feedstock is selected from the group consisting of waste restaurant frying oils, waste automotive oils, waste industrial greases, and waste industrial oils.

13. A process according to claim 1 wherein said anhydrous alcohol is selected from the group consisting of methanol, ethanol, propanol and butanol.

14. (canceled)

15. A process for producing alkyl esters useful as biofuels and lubricants, wherein said process comprises:
controllably heating a glyceride-containing feedstock to a temperature within the range of about 90° C. to about 100° C., and then dewatering said heated feedstock by the application of a negative pressure of about 110 mmHg(A) for about 60 minutes;
controllably combining said dewatered glyceride-containing feedstock with an alcohol to form a single-stage esterification reaction mixture, said alcohol containing therein a basic reaction catalyst;
separating a reaction product comprising alkyl esters from the reaction mixture containing therein a reaction by-product comprising alcohol containing therein the reaction catalyst, glycerol and glycerol-containing substances; and
controllably dewatering and then, purifying said reaction product.

16. A process according to claim 15 wherein said glyceride-containing feedstock is controllably dewatered under conditions comprising a temperature selected from the range of 50° C. to 100° C. and a negative pressure selected from the range of 99 mm Hg(A) to 760 mm Hg(A).

17. A process according to claim 15 wherein said dewatered glyceride-containing feedstock is cooled to a temperature selected from the range of 40° C. to 85° C. while providing a negative pressure selected from the range of 99 mm Hg(A) to 760 mm Hg(A), whereafter said cooled dewatered glyceride-containing feedstock is combined with said alcohol to form a reaction mixture, said alcohol containing therein said reaction catalyst, and controllably maintaining said reaction mixture for a time sufficient for esterification of the glycerides to occur.

18. A process according to claim 15 wherein said dewatered reaction product is purified by contacting said dewatered reaction product with an adsorbent material.

19. A process according to claim 18 wherein said adsorbent material is selected from the group consisting of silicas and clays.

20. A process according to claim 15 wherein said reaction by-product is further processed to separate said alcohol containing therein said reaction catalyst from said glycerol and glycerol-containing substances.

21. A process according to claim 20 wherein said separated alcohol containing therein said reaction catalyst is controllably dewatered.

22. A process according to claim 15 wherein said alcohol containing therein a reaction catalyst is the dewatered alcohol of claim 21.

23. A process according to claim 20 wherein said glycerol and glycerol-containing substances are further processed to separate and purify said glycerol and glycerol-containing substances.

24. A process according to claims 15 wherein said glyceride-containing feedstock comprises oils extracted from materials selected from the group consisting of plants, animals and fish.

25. A process according to claim 24 wherein said plant materials are selected from the group consisting of brassicas, legumes, maize, mallows, flax and palms.

26. A process according to claim 25 wherein said plant materials are seeds selected from the group consisting of mustard seeds, canola seeds and rape seeds.

27. A process according to claim 25 wherein said plant materials are soybean seeds.

28. A process according to claim 25 wherein said plant materials are corn seeds.

29. A process according to claim 25 wherein said plant materials are cotton seeds.

30. A process according to claim 25 wherein said plant materials are flax seeds.

31. A process according to claim 25 wherein said plant materials are palm nuts.

32. A process according to claim 23 wherein said glyceride-containing feedstock is selected from the group consisting of waste restaurant frying oils, waste automotive oils, waste industrial greases, and waste industrial oils.

33. A process according to claim 15 wherein said alcohol is selected from the group consisting of methanol, ethanol, propanol and butanol.

34. (canceled)

35. (canceled)
